(19) United States
(12) Patent Application Publication
(10) Pub. No.: US 2011/0027877 A1
(43) Pub. Date: Feb. 3, 2011

(54) RECIPIENT DEVICE AND METHOD TO PROTECT IN VITRO CULTURED EMBRYOS AND CELLS AGAINST ATOMOSPHERIC SHOCK

(76) Inventor: Ricardo Pimenta Bertolla, Sao Paulo-Sp (BR)

Correspondence Address: DARBY & DARBY P.C., P.O. BOX 770, Church Street Station, New York, NY, 10008-0770, US

(21) Appl. No.: 12/677,943

(22) PCT Filed: Oct. 6, 2008
(86) PCT No.: PCT/BR08/00302

§ 371 Date: Oct. 25, 2010

(30) Foreign Application Priority Data
Oct. 5, 2007 (BR) PI 0705219-7

(51) Int. Cl. C12N 5/07; C12M 1/00

(52) U.S. Cl. 435/325; 435/303.1

(57) ABSTRACT

A recipient device comprises a base (10) with a transparent bottom wall (11) and at least a sidewall portion (12) which defines, superiorly, a seat (5) for a removable and transparent lid (20), to be attached to the base (10), to be selectively switchable between a non-operant position, allowing fluid communication between the interior and exterior of the recipient device (R), and an operant position, hermetically sealing the recipient device (R) in an airtight manner, not allowing fluid communication between the interior and the exterior of said recipient device (R) which may be maintained inside an incubator, with the lid (20) in a non-operant position, until a control operation is desired, moment in which the lid (20) is switched to its operant position while still inside the incubator, maintaining the recipient device hermetically closed during its removal from the incubator.

### FIELD OF THE INVENTION

The present invention refers to a recipient device defined as a culture dish or bottle, or as a system to contain culture dishes or bottles, which may contain one or more embryos or cells to be cultured inside an incubator and which will periodically be submitted to control operations, mainly visual observations under a microscope or stereomicroscope, outside the incubator, without the occurrence of atmospheric shock to the cultured material once it is removed from within the incubator.

It also refers to a method developed to protect the cultured embryos and cells against atmospheric shock when they are removed from within the incubator for analysis of development.

### BACKGROUND OF THE INVENTION

Cell culture is based on maintenance of ideal temperature, media pH, and humidity conditions. Control of media pH is achieved through the sodium bicarbonate buffer system, which is based on an atmosphere containing high levels of carbon dioxide (CO₂). Some culture systems also control atmospheric Oxygen (O₂) and Nitrogen (N₂) levels, besides CO₂. This culture is performed in appropriate incubators, capable of maintaining controlled temperature, relative humidity, and atmospheric conditions.

During evaluation of the development of cell lineages or embryos it is necessary to remove the samples from inside the incubator in order to evaluate them under microscopy, for example. During this evaluation, a decrease in temperature and CO₂ levels under which the cells are being kept occurs.

Current systems prevent the temperature decrease satisfactorily through warm contact surfaces, but there is no method of maintaining, inside the culture device, an atmosphere akin to that from within the incubator during external culture conditions. Embryos and cells are usually cultured in dishes or bottles that allow atmospheric changes inside the incubator. However, there is the necessity to remove these culture dishes or bottles from within the incubators for observation of development. In that moment, gas change occurs between the dish or bottle and the environment, leading to atmospheric and temperature shock (and, thus, to oxidative stress), which may influence physiological development of the cells and embryos. These alterations may influence scientific experiments, cause loss in commercial systems, and decrease results in human infertility treatments.

### SUMMARY OF THE INVENTION

Due to the deficiency in the current solutions currently known in avoiding atmospheric shock to cultured embryos and cells when they are removed from within the incubator, our invention is aimed towards providing a recipient device of simple construction and handling and of relatively low cost which allows the maintenance, in a safe manner, of the same atmospheric conditions in its interior as those observed in the incubator, while also allowing the contents in its interior to be readily inspected.

The aforementioned objective is reached by providing a recipient device comprised of a base containing a transparent bottom wall and at least a sidewall portion with a top opening which defines a deposition site; and a removable lid to be attached to the base, over its top opening, at least partially formed of transparent material, and to be selectively switchable, when attached to the body, between a non-operant position, which allows fluid communication between the interior and the exterior of the recipient device, and an operant position, which seals the superior opening of the recipient device in an airtight manner and does not allow fluid communication between its interior and exterior.

The aforementioned constructs allows that the recipient device be easily sealed and shut in an airtight manner by the culture technician before removing it from within the incubator, and then easily opened when restored to inside the incubator, thus guaranteeing maintenance of the atmospheric conditions from the incubator to the cultured material throughout the controlling operations.

It is also an objective of this invention to promote a method to protect, against atmospheric shocks, in vitro cultured cells and embryos, in recipient devices maintained within the incubator in a controlled atmosphere, and to be periodically and momentarily removed from within the incubator for controlling operations.

This objective is achieved by a method which is comprised of the following steps:

a) place the embryo or cells inside the recipient device (culture dish or bottle) comprised of a base containing a transparent bottom wall and at least a sidewall portion with a top opening which defines a deposition site; and a removable lid to be attached to the base, over its top opening, at least partially formed of transparent material;

b) place the recipient device inside an incubator with a controlled atmosphere, maintaining the lid in a non-operant position, in which it remains attached to the base of the recipient device and allows fluid communication between the interior of the recipient device and the interior of the incubator;

c) switch the lid to an operant position, in which it remains attached to the base of the recipient device, seals the superior opening of the recipient device in an airtight manner and does not allow fluid communication between its interior and the interior of the incubator, immediately prior to the removal of the recipient device from within the incubator; and

d) remove the recipient, closed by the lid in an airtight manner, from within the incubator, in order to submit it to a culture control operation, and place it back inside the incubator as soon as this operation is finalized; and

e) switch the lid back to the non-operant position.

It may be observed that the invention promotes the decrease of the atmospheric shock to embryos and cells maintained in an in vitro culture system inside an incubator designed to maintain the recipients (culture dishes or bottles). The proposed solution allows gas exchange between the interior of the incubator and the interior of the recipient when in the non-operant lid mode, but does not allow any gas exchange between the interior of the recipient device and its exterior when the lid is switched to the airtight operant position, thus allowing the recipient device to be removed from within the incubator without alteration to its atmosphere, maintaining an atmosphere akin to that maintained inside the incubator.

The transparent material in the superior lid and in the bottom wall of the base allows observation of embryo or cellular development without the necessity to open the recipient device, thus preserving an adequate atmospheric condition to biologic structures.

### BRIEF DESCRIPTION OF THE DRAWINGS

The invention will be described in the following, referencing to the attached drawings, given solely as examples of possible configurations for the invention, in which:

FIG. 1 represents an exploded top view in perspective of the recipient device constructed according to a first configuration of the invention, illustrating the lid detached and apart from the base;

FIG. 2 represents a top plan view of the recipient device from FIG. 1, with the lid attached to the base;

FIGS. 3A and 3B represent diametric sections of the recipient device, cut through line III-III from FIG. 2 and illustrating the lid attached to the base, in the non-operant and operant positions, respectively;

FIG. 4 represents an exploded top view in perspective of the recipient device constructed according to a second configuration of the invention, illustrating the lid detached and apart from the base;

FIG. 5 represents a top plan view of the recipient device from FIG. 4, with the lid attached to the base and in a non-operant position;

FIG. 5A represents a longitudinal section of the recipient device, cut through line V-V from FIG. 5;

FIG. 6 represents a top plan view of the recipient device from FIG. 4, with the lid attached and in an operant position;

FIG. 6A represents a longitudinal section of the recipient device, cut through line VI-VI from FIG. 6; and

FIG. 6B represents a cross-section of the recipient device, cut through line VII-VII from FIG. 6, illustrated with the lid in an operant position.

### DETAILED DESCRIPTION OF THE INVENTION

As aforementioned and illustrated in the attached drawings, one of the objects in the present invention refers to a recipient device R, usually in the form of an embryo and cell culture dish or bottle, constructed in any appropriate material, preferably in disposable (nontoxic plastic) or autoclavable (glass and aluminum) material. The dimensions for the recipient device R may vary according to the culture characteristics, in that it may, for example, be constructed to hold three dishes with a 35 millimeter diameter, usually used in embryo culture. It may also be itself a culture dish, maintaining the characteristics of being an opened and closed system. One possible nontoxic plastic which may be used in the construction of said recipient device is polystyrene, which may be used for the whole recipient device R or for certain parts of it which require transparency for visual inspection.

Recipient device R is comprised of a base 10, cylindrical or prismatic, presenting a bottom wall 11 of transparent material, for example polystyrene or glass, and at least a sidewall portion 12 which may or may not be transparent, depending on the project of recipient device R.

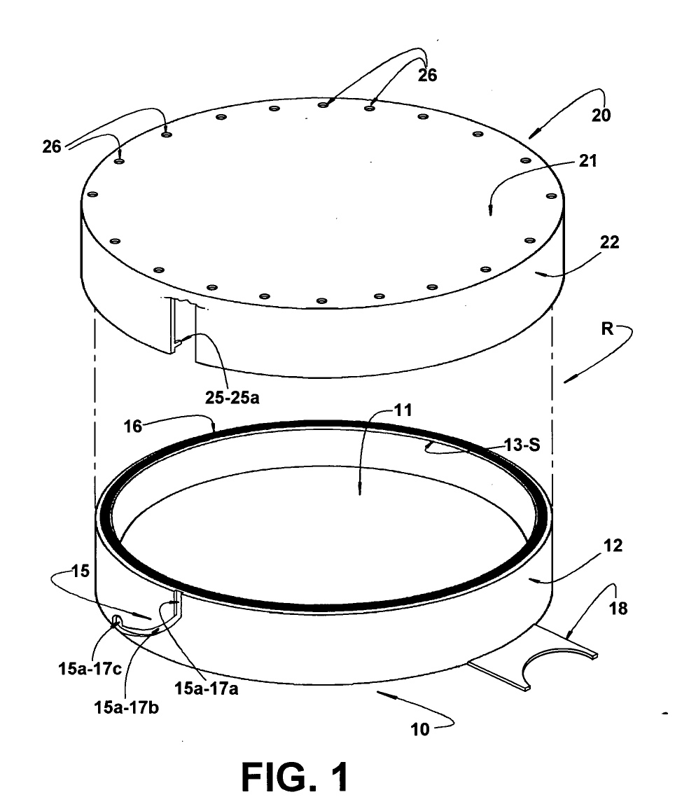

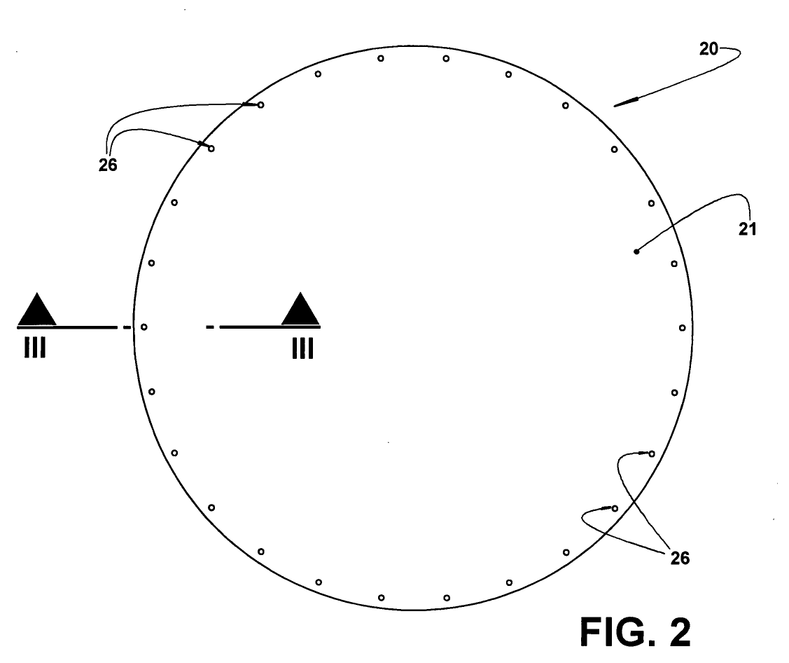

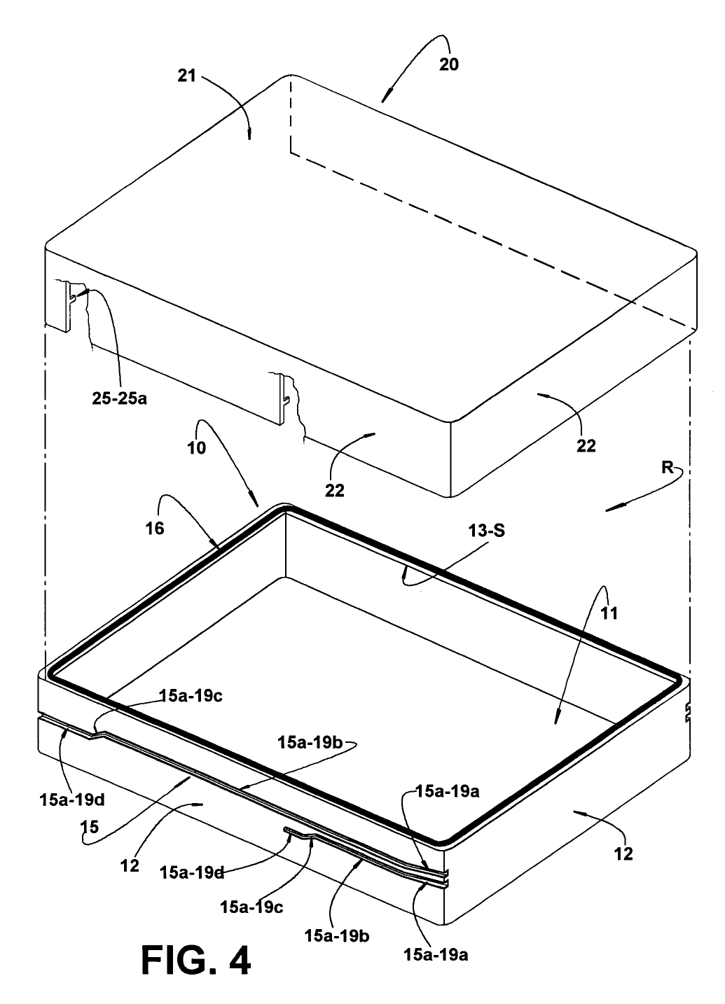

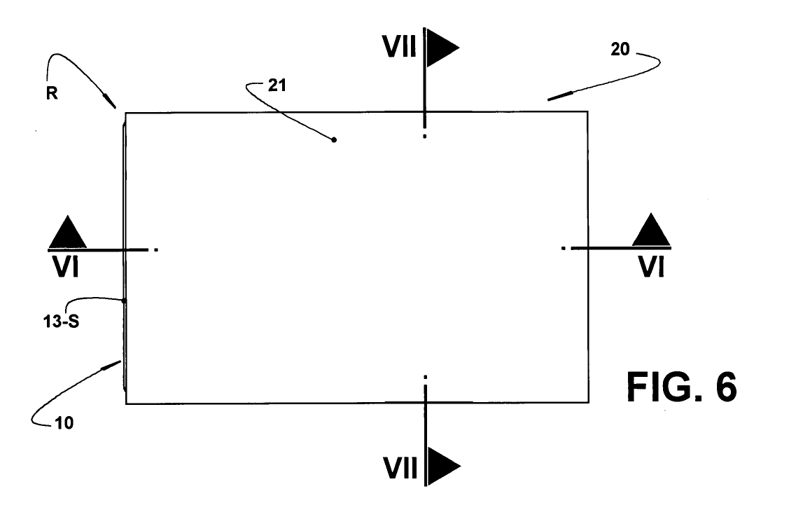

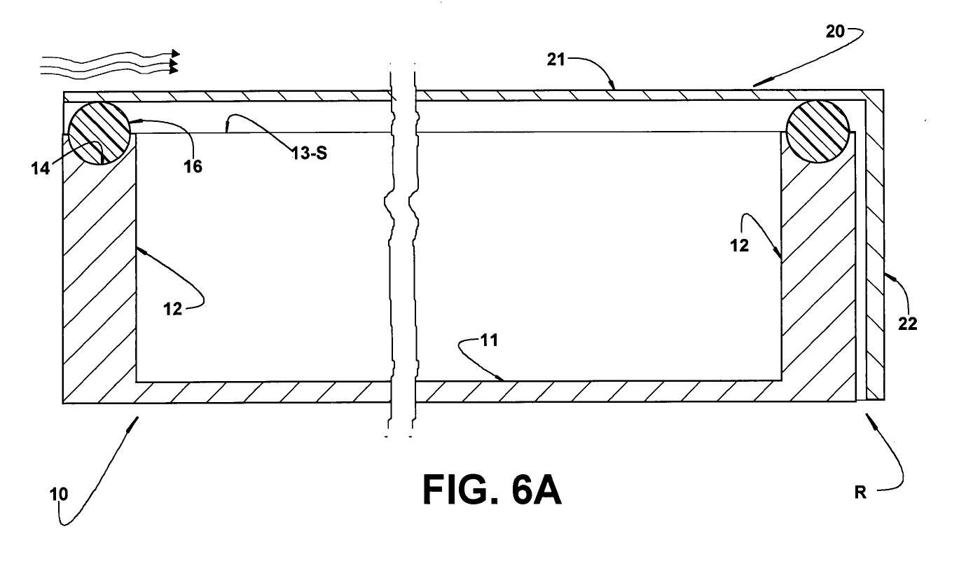

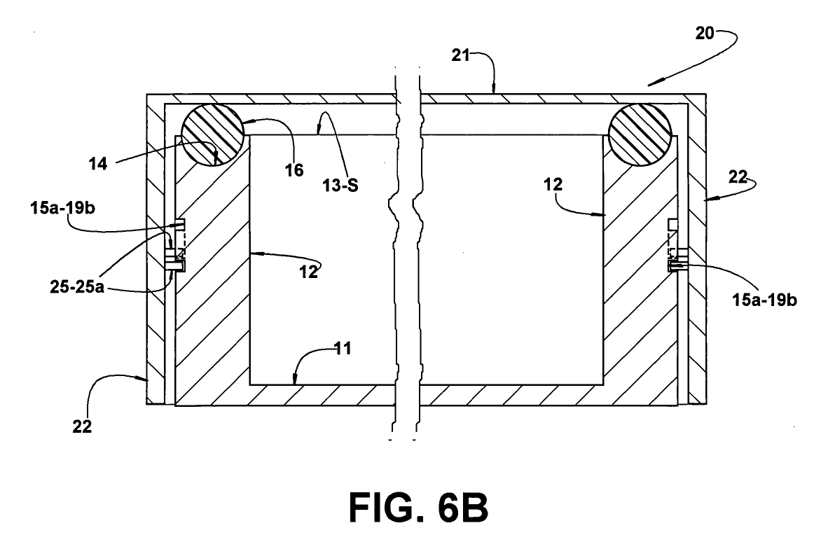

In the construct illustrated in FIGS. 1 through 3B, the recipient device R presents a cylindrical form with only a sidewall portion 12, while, in the construct illustrated in FIGS. 4 through 6B, the recipient device R presents a prismatic format with a rectangular base.

Independent of its format, recipient device R presents the sidewall 12 with a top opening 13 that defines a deposition site S for a lid 20 which is removable and attachable to the base 10, over its top opening 13, the lid being at least partially formed of transparent material and that, when attached to the base 10, it may be selectively displaceable by the technical operator between a non-operant position, which allows fluid communication between the interior and the exterior of the recipient, and an operant position, which seals the top opening 13 of recipient R in an airtight manner and does not allow fluid communication between the interior and the exterior of the recipient.

Also, independent on the format of the base 10 of recipient device R, the lid 20 forms a top panel 21, necessarily constructed of transparent material and to be set over the Seat S when the lid 20 is conducted to the operant position and to be removed from this Seat S when the lid 20 is conducted to the non-operant position.

The lid 20 also comprises at least a portion of the lateral face 22, which drops off from the top panel 21 and internally comprised of at least one pair of cam adapters 25, preferably in the form of pins 25a which project inward from their respective portion on the lateral face 22. On the other hand, each portion of the lateral face 12 of the base 10 is externally provided with two cams 15, generally as grooves 15a, around the lateral face 12 of base 10, turned outwards, and to which the cam adapters 25 are respectively and operationally associated through a sliding movement when the lid 20 is attached to the base 10, in that operational attachment of the cam adapters 25 to the cams 15 allow that de lid 20 be guided between a non-operant and an operant position. In the configuration illustrated in FIGS. 1 to 3B, the recipient device R has a bottom wall 11 with a circular outline and is provided with one set of a circumventing sidewall 12, with a superior border which supports a ring gasket 16, generally in elastic material and which is part of Seat S of lid 20 from base 10. In a similar manner, lid 20, in this configuration, presents a top panel 21, with a circular outline, carrying below it a peripheral ring crib 24, with a preferably concave transversal section, in a circular semi-arc, provided with a number of axial passing orifices 26, made through the cited superior panel 21.

It should be observed that the bottom wall 11 from base 10 and the superior panel of lid 20 should be constructed in transparent material in order to allow effortless visualization of the cultured material, when it is submitted to evaluation by light microscopy, for example, without removal of lid 20.

The transparent walls of base 10 and lid 20 may be made in colored material, capable of avoiding the passage of light wavelengths which may be harmful to biological structures, such as, for example, ultraviolet. This protection against potentially harmful light may be obtained through the building transparent material of base 10 and lid 20, or also by the application of a plastic film, not illustrated here, which functions as a filter. Thus, the recipient device R discussed here may be constructed in a manner which avoids exposition to deleterious luminosity and/or to promote bio-stimulation through an adequate construction of its base and lid walls. It should also be explained that bottom wall 11 of base 10 may be constructed with delimitations supporting borders for culture elements, avoiding that these latter should slide in the interior of recipient device R during the transport operations.

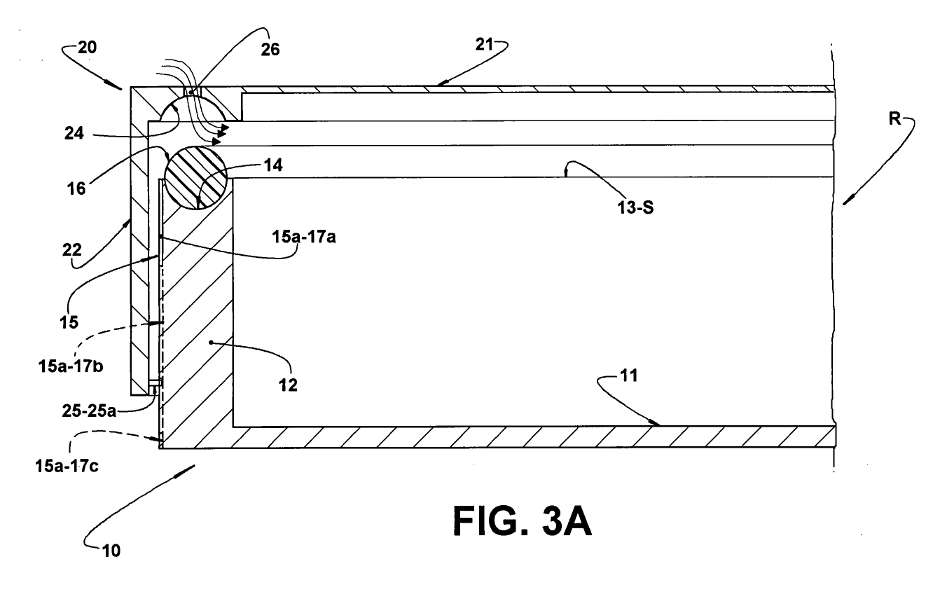

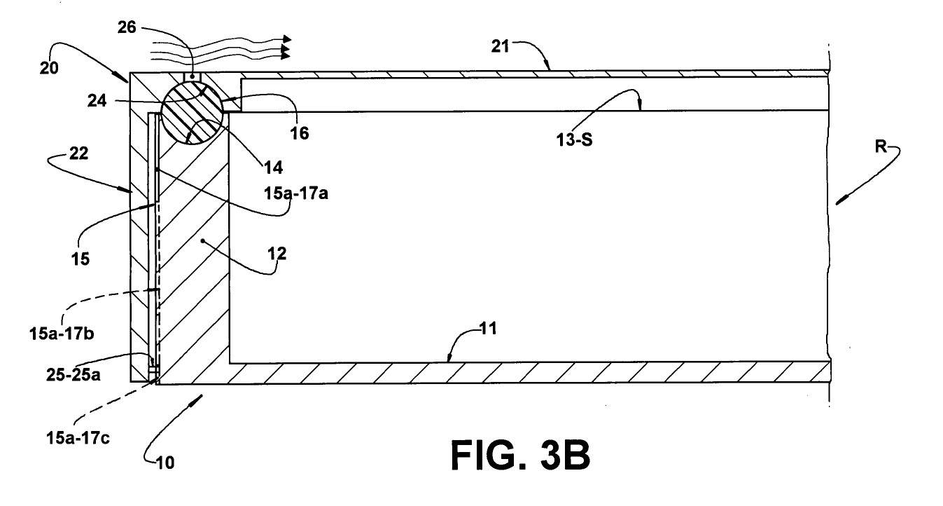

Also, as illustrated in FIGS. 1 to 3B, base 10 of the recipient device R may be constructed in a manner as to incorporate, inferiorly, two or more projecting radial brims to facilitate support of the technical operator's fingers in order to fix base 10 during the operations of opening and closing of lid 20 in this configuration, in which dislocation of lid 20 in relation to base 10 is achieved through a relative twist around the geometrical axis of the construct. According to this first configuration, the peripheral ring crib 24 of lid 20 is set on the sealing ring gasket 16 of base 10 when lid 20 is switched to its operant position, blocking the axial passing orifices 26 in an airtight manner, as illustrated in FIG. 3B. On the other hand, the peripheral ring crib 24 on lid 20 is axially removed from the sealing ring gasket 16 as illustrated in FIG. 3A when lid 20 is switched to its non-operant position, in order to allow fluid communication between the interior and the exterior of recipient device R, through the cited axial passing orifices 26, as represented by the arrows in FIG. 3A.

In this first configuration for recipient device R, each cam 15 possesses one groove 15a, positioned externally in the single set of sidewall 22 which circumvents base 10, while each cam adapter 25 is defined by a pin 25a which is projected inwards in a radius from the single set of sidewall 22 that circumvents lid 20, in a manner to cooperate with the respective grooves 15a, when lid 20 is attached to base 10. p Still according to the first configuration of the recipient device R, each groove 15a presents one end open towards the outer portion of the superior rim of the sidewall 12 that circumvents base 10, in a manner as to allow attachment of lid 20 to base 10, trough fitting and sliding of each pin 25a to a respective groove 15a. In this construct, each groove 15a presents an initial axial extension 17a, projecting downwards from its open end, and a second extension 17b which extends obliquely downwards from the initial extension 17a and which ends in a small extension retention 17c for the respective pin 25a when lid 20 is taken to its operant position by twisting it in relation to base 10.

As it may be observed, in the first construct for recipient device R in a cylindrical format, lid 20 is attached to base 10 by initially fitting a pair of pins 25a from lid 20 into the initial axial extension 18a of a respective groove 15a, dislocating the lid axially towards base 10, until pins 25a reach the inferior end of the initial axial extension 17a of groove 15a, in which situation lid 20 remains stably attached to base 10 in a non-operant condition or, in other words, in a condition in which its peripheral ring crib 24 remains axially removed from the sealing ring gasket 16, releasing passing orifices 26 and, thus, fluid communication between the interior and exterior of recipient device R as illustrated in FIG. 3A.

Lid 20 may then be displaced to its operant position by a simple twisting motion, enough so that pins 25a slide along the second extension 17b of the respective groove 15a, until they reach the small extension retention 17c, condition illustrated in FIG. 3B and in which the peripheral ring crib 24 from lid 20 is set and compresses the sealing ring gasket 16, blocking passing orifices 26 and, thus, sealing the interior of recipient R in an airtight manner. Displacement of lid 20 back to its non-operant position is achieved through twisting it in the opposite direction, in a manner that pins 25a slide ascendant along the second extension 17b of grooves 15a until they reach the junction between the second extension 17b and the initial axial extension 17a of grooves 15a, condition in which lid 20 is stabilized in its non-operant position but still attached to base 10 over the superior opening 13 of this latter.

Although not illustrated here, it should be understood that this first cylindrical configuration for recipient device R could be provided with valves throughout the superior panel 21 of lid 20 and throughout sidewall 12 which circumvents base 10, by means of one or more orifices disposed in a transversal plane to the longitudinal axis of recipient device R, these orifices aligned axially and unaligned, in a manner to permit closing of recipient device R in an airtight manner or fluid communication between this latter's interior and exterior through a corresponding twist of lid 20 in relation to base 10 in a similar constructive disposition to the various known arrangements present in the current state-of-the-art. However, because a high grade of airtight sealing is required device R in the operant condition of the lid, these orifices should be aligned in a condition that a ring gasket in elastic material, not illustrated, should be provided between the top panel 21 from lid 20 and the sidewall 12 circumventing base 10, acting between these two and involving each respective set of air passage orifices.

In the configuration illustrated in FIGS. 4 to 6B, the recipient device R presents a bottom wall 11 for base 10 in a rectangular outline, in which ends are incorporated four sets of sidewall 12, parallel and opposite two by two, and presenting a top rim which carries a sealing ring gasket 16 which is part of the Seat S of lid 20 on base 10, in that the top panel 21 on lid 20 presents a corresponding rectangular outline to be set against the sealing ring gasket 16 when the lid 20 is conduced to the operant position, in a manner that seals recipient device R in an airtight manner.

Lid 20 is displaceable in a manner that allows its top panel 21 to be axially removed from sealing ring gasket 16 when lid 20 is conduced to its non-operant position, in order to allow fluid communication between the interior and the exterior of the recipient device through a space defined between top panel 21 and sealing ring gasket 16.

In this configuration for recipient device R in a prismatic box format, lid 20 is comprised of two sets of lateral face 22 which drop off from top panel 21, opposite to each other, and each cam 15 is comprised of a pair of grooves 15a, externally in a respective portion of sidewall 12 of base 10. Each cam adapter 25 is defined by a pair of pins 25a which project inwards from each portion of the lateral face 22 of lid 20, to cooperate with their respective pair of groove 15a, when lid 20 is attached to base 10.

In this second prismatic configuration for recipient device R, each groove 15a presents one end opened towards the extreme rim of the respective sidewall 12 of base 10, in a manner that permits the attachment of lid 20 to base 10 through a linear sliding motion of the lid 20 in relation to the base 10. Each groove 15a preferably presents an entrance extension 19a, projecting from the open end with a slight declining inclination, a positioning extension 19b which extends in parallel to the plane of the sealing ring gasket 16, a setting portion 19c which extends inclined downwards from positioning extension 19b and a retention extension 19d to receive and maintain respective pin 25a when lid 20 is conduced to the operant position by a linear sliding movement (or any other form of displacement which maintain the characteristics of operant and non-operant) in relation to base 10. In this construct grooves 15a of each pair of grooves are parallel among themselves and positioned in different levels in each respective portion of sidewall 12 of base 10, the same alterations in level occurring in relation to the respective pins 25a built in each set of sidewall 22 which drop off from lid 20.

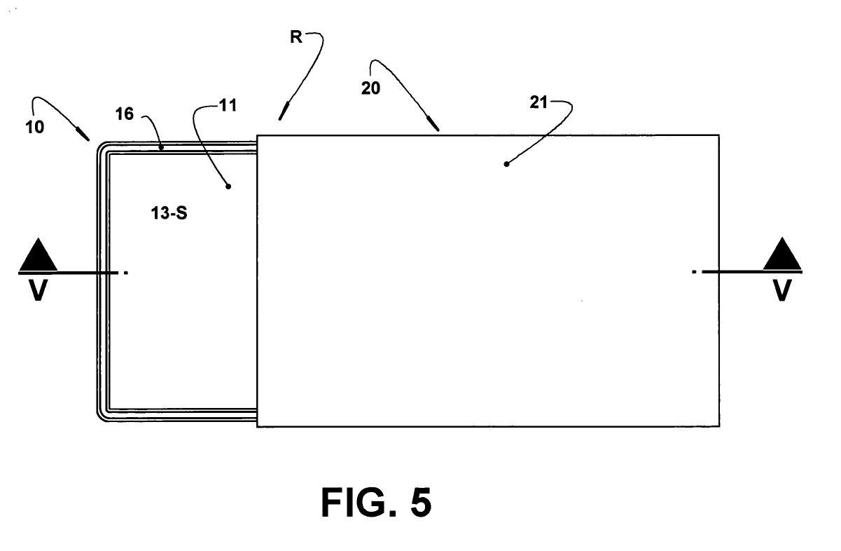

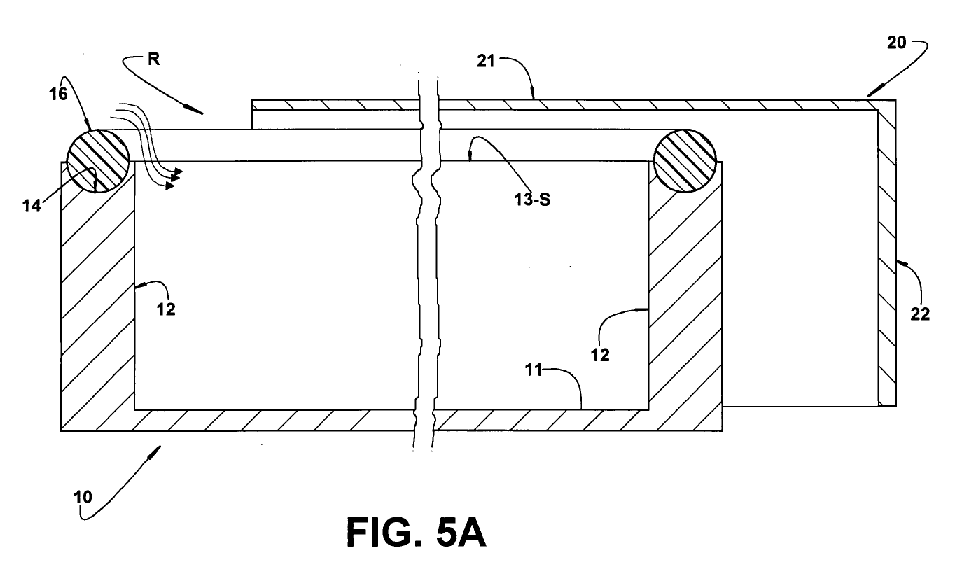

As it may be observed, in the prismatic box configuration, dislocation of lid 20 between the operant and non-operant positions is performed in a linear path. When lid 20 is conduced to its non-operant position, as illustrated in FIGS. 5 and 5A, it occupies a position partially outside base 10, allowing fluid communication between the interior and exterior of recipient R not only in the space defined between top panel 21 of lid 20 and sealing ring gasket 16, but also by the opening formed by the linear displacement of lid 20 in relation to base 10.

Independent of a cylindrical or prismatic format for base 10 applied to recipient device R, the superior rim of base 10 is provided with a groove 14 in the interior of which is placed sealing ring gasket 16, the sealing ring being held inside groove 14 performed by different manners, such as, for example, by conformation of the transversal profile of groove 14 and/or by the application of a retaining element, for example, an adhesive.

As may be understood by the description above, the proposed construction for recipient R allows the realization of a method to protect cultured embryos and cells against atmospheric shocks when removed from inside the incubators. The method proposed in the present invention is comprised of a first measure, which is to place the embryo or cell inside a recipient device R (or to place the embryo or cell inside a culture bottle or dish and to place this bottle or dish inside recipient device R) in a cylindrical, rectangular prismatic or any other forma, as previously described. After placing the embryo inside recipient device R, lid 20 of this latter is attached to its base 10, through fitting two or more pins 25a into their respective grooves 15a, independent of the format for recipient device R. After being replaced within an incubator (not illustrated), of any build known at the state-of-the-art and presenting a controlled atmosphere, lid 20, attached to recipient device R, is displaced to its non-operant position and the incubator is quickly closed in order to maintain the atmosphere conditions. In this condition, recipient device R remains inside the incubator with the lid in a non-operant position, permitting fluid communication between the interior of recipient device R and the interior of the incubator, generally with a controlled atmosphere.

Before removing a recipient device R from within the incubator for evaluation of development of the samples, the operating user should dislocate lid 20 to an operant position so that it may seal the recipient in an airtight manner, thus not allowing any fluid communication between the interior of recipient device R and its exterior, be that exterior the interior or exterior of the incubator. If the recipient device is of a cylindrical format, dislocation of the lid should be performed through a controlled twist, while if it is in the prismatic box, dislocation should be performed in a linear manner, as previously described. After the recipient device R is hermetically closed by lid 20, it may then be removed from inside the incubator to be submitted to culture control operations, and then replaced inside the incubator as soon as this operation is finished. After replacing the recipient device R inside the incubator, lid 20 is once again displaced into a non-operant position illustrated in FIGS. 3A, 5, and 5A, in that fluid communication between the interior of the recipient device and the interior of the incubator is reestablished.

The solution proposed by this invention allows that the technical operator may easily move the lid 20 inside the incubator, in a way as to conduce it to an operant position, hermetically shut, before any operation of removal of recipient devices R from inside the incubator for the periodic inspections, thus not allowing alterations in its internal atmosphere which could alter the conditions on relation to those observed inside the incubator and that tend to produce harmful effects to cultured cells or embryos. Although only two examples of configurations were illustrated here, it should be understood that alterations to form and disposition of the elements may be applied, without these alterations causing the final construct to be different in constructive and operational concept defined in the claims that follow this report.

1-20. (canceled)

21. Recipient device for culturing embryos and cells wherein it is comprised of:
a base presenting a transparent bottom wall, at least a sidewall portion provided with a top opening defining a seat; a lid that is removable and attachable to the base, over the latter's top opening, at least partially formed of transparent material and to be selectively switchable, when attached to the base, between a non-operant position, allowing fluid communication between the interior and the exterior of the recipient device, and an operant position, which hermetically seals the top opening in an airtight manner, said lid comprising: a top panel to be set against the seat when the lid is conduced to the operant position, and removed from the seat when the lid is conduced to its non-operant position; and at least one set of lateral face, dropping off from the top panel, and carrying, internally, at least one pair of cam adapters, being the base provided with at least a sidewall portion externally provided with cams to which the cam adaptors are operationally and respectively associated when the lid is switched to its condition attached to the base, in a way as to guide the movement of the lid between its operant and non-operant position; said recipient device being wherein it presents a round bottom wall and a set of sidewall circumventing the bottom wall, with a top rim carrying a sealing ring gasket defining seat for the lid, built of a round top panel carrying, inferiorly, a peripheral ring crib provided with a number of passing axial orifices through the top panel, being the said peripheral ring crib set against the sealing ring gasket, when the lid is conduced to its operant position, in a way as to hermetically block the passing axial orifices, and axially removed from the sealing ring gasket when the lid is conduced to its non-operant position, allowing fluid communication between the interior and exterior of the recipient device through the said passing axial orifices.

22. Recipient device, according to claim 21, wherein the lid presents a lateral face circumventing it, each cam being comprised of a groove, built externally to the sidewall circumventing the base and presenting one end open towards the outer portion of the superior rim of the sidewall, in a manner as to allow attachment of the lid to the base, trough fitting and sliding of each pin to a respective groove, each cam adapter being defined by a pin which projects inwards from the lateral face circumventing the lid, in order to cooperate with its respective groove when the lid is attached to the base.

23. Recipient device, according to claim 22, wherein each groove presents an initial axial extension, projecting downwards from its open end, and a second extension which extends obliquely downwards from the initial extension and which ends in a small extension retention for the respective pin when the lid is taken to its operant position by twisting it in relation to base.

24. Recipient device for culturing embryos and cells wherein it is comprised of: a base presenting a transparent bottom wall, at least a sidewall portion provided with a top opening defining a seat; a lid that is removable and attachable to the base, over the latter's top opening, at least partially formed of transparent material and to be selectively switchable, when attached to the base, between a non-operant position, allowing fluid communication between the interior and the exterior of the recipient device, and an operant position, which hermetically sealing the top opening in an airtight manner; said lid comprising: a top panel to be set against the seat when the lid is conducted to the operant position, and removed from the seat when the lid is conduced to its non-operant position; and at least one set of lateral face, dropping off from the top panel, and carrying, internally, at least one pair of cam adapters, being the base provided with at least a sidewall portion externally provided with cams to which the cam adaptors are operationally and respectively associated when the lid is switched to its condition attached to the base, in a way as to guide the movement of the lid between its operant and non-operant position; said recipient being wherein it presents a rectangular bottom wall and four sets of sidewall, parallel and opposite two by two, and presenting a top rim which carries a sealing ring gasket which defines the seat of the lid on the base, in that the lid is comprised of a rectangular top panel to be set against the sealing ring gasket when the lid is conduced to the operant position, in a manner that seals the recipient device in an airtight manner, and axially removed from the sealing ring gasket when the lid is conduced to its non-operant position, in a way that allows fluid communication between the interior and exterior of the recipient device through the space defined between the top panel and the sealing ring gasket.

25. Recipient device, according to claim 24, wherein the lid is comprised of two sets of lateral face which drop off from top panel, opposite and parallel to each other, each cam being comprised of a pair of grooves, externally in a respective portion of the sidewall of the base, each groove presenting one end opened towards the extreme rim of the respective sidewall of the base, in a manner that permits attachment of the lid to the base through a linear sliding motion of the lid in relation to the base; each cam adapter being defined by a pair of pins which project inwards from each portion of the lateral face of the lid, in order to cooperate with their respective pair of grooves, when the lid is attached to the base.

26. Recipient device, according to claim 25, wherein each groove presents an entrance extension, projecting from the open end with a slight declining inclination, a positioning extension which extends in parallel to the plane of the sealing ring gasket, a setting portion which extends inclined downwards from the positioning extension and which ends in a retention extension to receive and maintain the respective pin when the lid is conduced to the operant position by a linear sliding movement in relation to the base.

27. Recipient device, according to claim 26, wherein the grooves of each pair of grooves are parallel among themselves and positioned in different levels in each respective portion of the sidewall of the base, the same alterations in level occurring in relation to the respective pins.

28. Recipient device, according to either claim 21, wherein the top rim of the base is provided with a groove in the interior of which is placed a sealing ring gasket.

29. Method to protect, against atmospheric shocks, cultured embryos and cells in recipient devices maintained within the incubator in a controlled atmosphere, and to be periodically and momentarily removed from within the incubator for controlling operations, being said method wherein it comprises of the following steps:
a) place the embryo or cells inside the recipient device or in a culture dish or bottle which is placed inside the recipient device, said recipient device comprised of a base containing a transparent bottom wall and at least a sidewall portion with a top opening which defines a deposition site; and a removable lid at least partially formed of transparent material;
b) place the recipient device inside an incubator with a controlled atmosphere, maintaining the lid in a non-operant position, in which it remains attached to the base of the recipient device and allows fluid communication between the interior of the recipient device and the interior of the incubator;
c) switch the lid to an operant position, in which it remains attached to the base of the recipient device, hermetically sealing the superior opening of the recipient device in an airtight manner and does not allow fluid communication between its interior and the interior of the incubator, immediately prior to the removal of the recipient device from within the incubator; and
d) remove the recipient device, hermetically closed by the lid, from within the incubator, in order to submit it to a culture control operation, and place it back inside the incubator as soon as this operation is finalized; and
e) switch the lid back to the non-operant position.

30. Method, according to claim 29, wherein the recipient device is laterally and externally provided with cams, being the lid internally provided with cam adapters, the cams and cam adapters defining the attachment of the lid to the base and guiding the dislocation of the lid in relation to the base between the operant and non-operant positions.

31. Method, according to claim 30, wherein the dislocation of the lid is defined by a certain twist of the lid in relation to the base.

32. Method, according to claim 10, wherein the dislocation of the lid is produced in a linear direction, parallel to the top opening of the base.

33. Method, according to claim 29, wherein the fluid communication between the interior of the recipient device and the internal atmosphere of the incubator is achieved through passing orifices built in the lid.

34. Method, according to claim 29, wherein the fluid communication between the interior of the recipient device and the internal atmosphere of the incubator is achieved through a space defined between the seat and the lid, when the latter is in its non-operant position.

35. Recipient device, according to either claim 24, wherein the top rim of the base is provided with a groove in the interior of which is placed a sealing ring gasket.
